(19) United States
(12) Patent Application Publication
(10) Pub. No.: US 2009/0030079 A1
(43) Pub. Date: Jan. 29, 2009

(54) USES OF TRIENTINE AND PENICILLAMINE AS COUNTERMEASURES TO METAL CONTAMINATION

(75) Inventors: Barry Levinson, Highland Park, NJ (US); Michael Wells, Skillman, NJ (US)

Correspondence Address: FOLEY AND LARDNER LLP;SUITE 500, 3000 K STREET NW, WASHINGTON, DC, 20007, US

(73) Assignee: Aton Pharma, Inc.

(21) Appl. No.: 12/180,130

(22) Filed: Jul. 25, 2008

Related U.S. Application Data
(60) Provisional application No. 60/952,482, filed on Jul. 27, 2007.

(51) Int. Cl. A61K 31/197; A61K 31/132

(52) U.S. Cl. 514/562; 514/674

(57) ABSTRACT

Methods are provided for the alleviation, prevention and treatment of negative effects of overexposure to metal contaminants. Subjects exposed to a metal contaminant can be treated using trientine and/or penicillamine or salts, esters, solvates thereof. In addition, communities can protect its members by securing sufficient quantities of such countermeasures.

### CROSS-REFERENCE TO RELATED APPLICATION

This application claims the benefit of U.S. Provisional Application No. 60/952,482, filed Jul. 27, 2007, which is incorporated by reference in its entirety.

### TECHNICAL FIELD

This invention relates in general to the alleviation, prevention and treatment of negative effects of overexposure to metal contaminants and, in particular, to the uses of trientine and/or penicillamine as a countermeasure to metal contamination.

### BACKGROUND

Metal contaminants such as heavy metals pose danger and cause damage to wildlife and livestock, as well posing a health risk to mammals, including humans. It has been known that heavy metal contaminants form complexes upon absorption in human or animal bodies. These complexes then bind to essential amino acids, precipitate proteins, inhibit enzymes, or enter cells directly, often causing cell deterioration or even death. These effects are often greatly magnified if, in addition, a radioactive isotope of the metal is involved. In such cases, accumulation of amounts of metal, that would be otherwise not overly toxic, is debilitating or lethal due to the irradiation of key cells or organs.

Sources of metal contaminants include contaminated water, contaminated wildlife such as fish, paints, and industrial manufacturing processes. Military or terrorist use of radiological dispersal devices (RDDs) such as dirty bombs purposefully disseminate radioactive materials, almost all of them metals, causing radiation contamination as well as metal contamination, with concomitant physical injury. Radiation contamination results when a radioisotope, in the form of a gas, liquid, or solid, is released into the environment and then ingested, inhaled, or deposited on the body surface, or enters the body via a wound caused by radioactive shrapnel.

Trientine hydrochloride (SYPRINE®, ATON PHARMA, INC.) is a known compound, approved by the Food and Drug Administration for treating Wilson's disease. Wilson's disease (hepatolenticular degeneration) is an autosomal inherited metabolic defect resulting in an inability to maintain a near-zero balance of copper. Excess copper accumulates possibly because the liver lacks the mechanism to excrete free copper into the bile. Hepatocytes store excess copper, often leading to hepatitis or acute liver failure, and when their capacity is exceeded copper is released into the blood and is taken up into extrahepatic sites. When copper accumulates in the brain, it causes neurological damage and symptoms. Wilson's disease is treated with a low copper diet and the use of chelating agents, like trientine, that bind copper to facilitate its excretion from the body.

Penicillamine (CUPRIMINE®, ATON PHARMA, INC.) is another known compound that is also approved by the Food and Drug Administration for the removal of excess copper in patients with Wilson's disease. It also is used to reduce cystine excretion in cystinuria and to treat patients with severe, active rheumatoid arthritis that is unresponsive to conventional therapy.

U.S. Pat. No. 6,441,009 has disclosed agents and methods for preventing and treating heavy metal exposure and toxicity, the disclosure of which is hereby incorporated by reference.

As alleviation, prevention and treatment of the negative effects of metal contamination are continuing concerns, further developments are needed to meet the demands of subjects suffering such negative effects, especially on a large scale.

### SUMMARY

In one aspect, chelating agents such as trientine, penicillamine, their derivatives and pharmaceutically acceptable salts and solvates (and esters in the case of penicillamine) are used in the prevention and treatment of mammals suffering from one or more negative effects of overexposure to a metal contaminant. The metal contaminant, which can comprise one or more metals, includes at least one metal selected from the group consisting of a metal from Group IA, IIA, VIIB, VIIB, VIII, IB, IIB, IIIA, IVA, VA, VIA, Lanthanide Series, and Actinide Series of the periodic table, wherein the at least one metal is other than copper. A therapeutically effective amount of trientine or its pharmaceutically acceptable salt or solvate is administered to the affected mammal.

In some embodiments, a therapeutically effective amount of trientine or its pharmaceutically acceptable salt or solvate is administered to mammals suffering from one or more negative effects of overexposure to a metal selected from the group consisting of mercury, nickel, bismuth, palladium, zinc, cadmium, lead, cobalt, chromium, iron, silver, and cesium. In some embodiments, the metal contaminant is palladium or cobalt. The metal contaminant may be any metal whose presence in excessive amounts in the body of a mammal may be undesirable, detrimental, or otherwise poses an unacceptable risk to the short- or long-term health of the mammal. In one embodiment the metal contaminant is a radioactive isotope such as a radioactive isotope of a metal selected from the group consisting of americium, californium, cobalt, iridium, palladium, plutonium, polonium, and uranium. In one embodiment the radioactive isotope is cobalt-60 and/or polonium-210.

The administration of trientine or its pharmaceutically acceptable salt or solvate may be carried out enterally or parenterally. The dosage depends on the severity of the metal contamination and may range from about 4 mg of free base per kg of mammal per day to about 25 mg of free base per kg of mammal per day. In one specific embodiment, an effective amount of trientine is administered as its hydrochloride salt.

In some embodiments, trientine or its pharmaceutically acceptable salt or solvate is administered to the affected mammal sequentially or concomitantly with an effective amount of penicillamine or its pharmaceutically acceptable salt, ester, or solvate. Penicillamine or its pharmaceutically acceptable salt, ester, or solvate may be administered in a dose ranging from about 2 mg of penicillamine per kg of mammal per day to about 60 mg of penicillamine per kg of mammal per day. In some specific embodiments, effective amounts of trientine hydrochloride and D-penicillamine are co-administered.

In still other embodiments, a therapeutically effective amount of trientine or its pharmaceutically acceptable salt or solvate is administered to an affected mammal which is overexposed to at least two metals. For example, the at least two metals may include indium or polonium. In some embodiments, therapeutically effective amounts of trientine and penicillamine, or their respective pharmaceutically acceptable salts, esters or solvates, are administered to an affected mammal which is overexposed to at least two metals.

The invention also contemplates the use of trientine or its pharmaceutically acceptable salt or solvate in the treatment of a community of individuals against the negative effects of overexposure to metal contamination on a large scale. In this aspect, a quantity of dosage forms of trientine or its pharmaceutically acceptable salt or solvate is provided that is sufficient to treat within a week or less of such overexposure every member of a community numbering between 5,000 and 1,000,000 individuals. In some instances, a quantity of dose forms of penicillamine or its pharmaceutically acceptable salt, ester, or solvate is provided that is sufficient to treat every exposed member (or at least the great majority of exposed members) of the community.

This invention further contemplates stockpiling trientine, or its pharmaceutically acceptable salt or solvate in preparing a community against the negative effects of overexposure to metal contamination on a large scale. In this aspect, a quantity of dosage forms of trientine or its pharmaceutically acceptable salt or solvate is stockpiled sufficient to treat within a week or less of such overexposure every member of the community. The quantity of dose forms of trientine or its pharmaceutically acceptable salt or solvate may be stockpiled sufficient to prepare a community with members ranging between 5,000 and 1,000,000 individuals. In some instances, a quantity of dose forms of penicillamine or its pharmaceutically acceptable salt, ester, or solvate are stockpiled. In some instances, penicillamine and trientine are administered together in a single combination dosage form.

In another aspect, pharmaceutical dosages are provided that comprise an effective amount of trientine or its pharmaceutically acceptable salt or solvate and an effective amount of penicillamine or its pharmaceutically acceptable salt, ester or solvate and optionally one or more pharmaceutically acceptable carriers.

In another aspect, methods of treating a mammal suffering from one or more negative effects of overexposure to polonium are provided that comprise administering to the affected mammal a therapeutically effective amount of penicillamine or a pharmaceutically acceptable salt, ester, or solvate thereof. In some aspects the administration is carried out enterally or parenterally. In other aspects the penicillamine or a pharmaceutically acceptable salt or solvate thereof is administered in a dose ranging from about 2 mg of penicillamine per kg of mammal per day to about a 60 mg of penicillamine per kg of mammal per day.

### BRIEF DESCRIPTION OF THE FIGURES

FIG. 1 shows the radioisotope content of cobalt-60 in various organs in a rat 4 days after exposure to cobalt-60 and subsequent administrations of penicillamine or trientine.

FIG. 2 shows the radioisotope content of polonium-210 in various organs in a rat 5 days after exposure to polonium-210 and subsequent oral administrations of penicillamine or trientine.

### DETAILED DESCRIPTION

Various embodiments of the invention are described hereinafter. It should be noted that the specific embodiments are not intended as an exhaustive description of the invention or as a limitation on the scope of the invention. One aspect described in conjunction with a particular embodiment of the present invention is not necessarily limited to that embodiment and can be practiced with any other embodiment(s) of the invention.

In one embodiment, methods are provided for treating mammals suffering from negative effects of overexposure to metal contaminants. The metal contaminants may be one or more metals from Group IA, IIA, VIIB, VIIB, VIII, IB, IIB, IIIA, IVA, VA, VIA, Lanthanide Series, or Actinide Series of the periodic table, other than copper. The invention contemplates administration to the affected mammals of a therapeutically effective amount of trientine or its pharmaceutically acceptable salt or solvate.

Trientine is N,N′-bis (2-aminoethyl)-1,2-ethanediamine. It has the following structural formula (I):

NH₂(CH₂)₂NH(CH₂)₂NH(CH₂)₂NH₂ (I)

Trientine is a known chelating compound and is commercially available from, for example, Aton Pharma Inc., Lawrenceville, N.J. An exemplary pharmaceutically acceptable salt, trientine hydrochloride (SYPRINE®, ATON PHARMA, INC.), is N,N′-bis (2-aminoethyl)-1,2-ethanediamine dihydrochloride. Trientine is a white to pale yellow crystalline hygroscopic powder having a molecular weight of 219.2. It is freely soluble in water, soluble in methanol, slightly soluble in ethanol, and insoluble in chloroform and ether. The term “trientine” as used herein refers to both trientine free base and its pharmaceutically acceptable salt or solvate unless otherwise indicated.

In general, a “therapeutically effective” amount of a chelating agent can be determined by inhibition or mitigation of metal contaminant toxicity or promotion of its excretion from the body. The appropriate dosage will of course vary depending upon, for example, the type, severity and timing of the contamination, as well as on the mode of administration.

Trientine is administered to affected mammals in an amount effective to inhibit or mitigate metal contaminant toxicity or to promote its excretion from the body. Depending on the severity of metal contamination, trientine may be administered in a dose ranging from about 4 mg of free base per kg of mammal body weight per day to about 25 mg of free base per kg of mammal body weight per day. It is recommended that affected mammals are treated with effective amounts of trientine for a minimum of about seven consecutive days, or until suitable tests such as blood tests or urinalysis indicate that the undesirable levels of the metal contaminant have substantially subsided, or have otherwise fallen below a level deemed excessive or a threat to the subject's short- or long-term health.

Trientine may be administered by any pharmaceutically acceptable means and in any pharmaceutically acceptable form. For example, trientine may be administered orally in the form of either liquid or solid. Trientine may be in the form of solution, suspension, tablet, capsule, oral quick dissolve, sachet or sprinkle. For oral administration, trientine is preferably combined with one or more pharmaceutically acceptable excipients, fillers and/or diluents. Tablets or pills may be coated by conventional techniques to control disintegration and absorption of trientine in the gastrointestinal tract.

Trientine also may be administered parenterally (e.g., intravenously, intramuscularly or subcutaneously). For parenteral administration, trientine is preferably dissolved in a suitable solvent, forming a solution which may be injected.

By way of example, trientine hydrochloride (SYPRINE®) is available as 250 mg capsule for oral administration. Capsules of trientine contain gelatin, iron oxides, stearic acid, and titanium dioxide as inactive ingredients.

In some instances, trientine may be administered sequentially or concomitantly with other chelating agents such as penicillamine or its pharmaceutically acceptable salt, ester, or solvate.

The pharmaceutical form of penicillamine is 3-mercapto-D-valine (D-penicillamine). It is a white or practically white, crystalline powder, freely soluble in water, slightly soluble in alcohol, and insoluble in ether, acetone, benzene, and carbon tetrachloride. Penicillamine has the following structural formula (II):

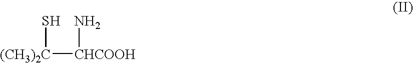

Penicillamine is commercially available from, for example, Aton Pharma Inc., Lawrenceville, N.J. (CUPRIMINE®). The term “penicillamine” as used herein refers to both penicillamine free base (or zwitterionic form) and its pharmaceutically acceptable salt, ester, or solvate unless otherwise indicated.

Penicillamine may be administered in a dose ranging from about 2 mg of penicillamine per kg of mammal body weight per day to about 30 mg of penicillamine per kg of mammal body weight per day.

Penicillamine may be administered by any pharmaceutically acceptable means and in any pharmaceutically acceptable form. For example, penicillamine may be administered orally in the form of either liquid or solid. Penicillamine may be in form of solution, suspension, tablet, capsule, oral quick dissolve, sachet or sprinkle. For oral administration, penicillamine is preferably combined with one or more pharmaceutically acceptable excipients, fillers and/or diluents. Tablets or pills may be coated by conventional techniques to control disintegration and absorption of penicillamine in the gastrointestinal tract.

Penicillamine also may be administered parenterally (e.g., intravenously, intramuscularly or subcutaneously). For parenteral administration, penicillamine is dissolved in a suitable solvent, forming a solution which may be injected.

By way of example, penicillamine (CUPRIMINE®) is available as a 250 mg capsule for oral administration. Capsules of penicillamine can contain gelatin, lactose, magnesium, stearate, and titanium dioxide as inactive ingredients.

Penicillamine is administered to affected mammals in an amount effective to inhibit or mitigate metal contaminant toxicity. Depending on the severity of metal contamination, penicillamine may be administered in a dose ranging from about 2 mg per kg of mammal body weight per day to about 25 mg of free base per kg of mammal body weight per day. It is recommended that affected mammals are treated with effective amounts of penicillamine for a minimum of about seven consecutive days, or until suitable tests such as blood tests or urinalysis indicate that the undesirable levels of the metal contaminant have substantially subsided, or have otherwise fallen below a level deemed excessive or a threat to the subject's short- or long-term health.

This invention is further directed to a pharmaceutical dosage form comprising a therapeutically effective amount of trientine or its pharmaceutically acceptable salt or solvate and a therapeutically effective amount of penicillamine or its pharmaceutically acceptable salt, ester or solvate and optionally one or more pharmaceutically acceptable carriers. The pharmaceutical dosage form may be in any suitable form such as solution, suspension, tablet, capsule, oral quick dissolve, sachet or sprinkle. The pharmaceutical dosage form may be used for treating patients suffering from metal contamination including radioactive metal contamination.

As indicated above, the invention is suitable for use in treatment of patients suffering from metal contamination including heavy metal contamination. Such metals include metals from Group IA, IIA, VIIB, VIIB, VIII, IB, IIB, IIIA, IVA, VA, VIA, Lanthanide Series, and Actinide Series of the periodic table. Examples of such metals include chromium, manganese, iron, cobalt, nickel, copper, zinc, strontium, palladium, silver, cadmium, indium, cesium, iridium, mercury, thallium, lead, bismuth, polonium, radium, cerium, uranium, plutonium, americium, and californium. While the invention is not limited to any particular theory, it is believed that metal contaminants form complexes with chelating trientine and/or penicillamine. The chelated metal complexes are inactivated and excreted through the urine or feces.

The invention is suitable for use as a countermeasure to heavy metal contamination such as for example palladium, mercury, bismuth, copper, iridium, nickel, zinc, cadmium, lead, cobalt, and silver.

By way of example, TABLE 1 provides in vitro binding stability constants (log K) for trientine and penicillamine with some exemplary heavy metals (Critically Selected Stability Constants of Metal Complexes, NIST Std. Ref. Database 46, December 1997; Critical Stability Constants, A. E. Martell & R. M. Smith, Vols. 2, 5, 6 (NY: Plenum, 1974, 1982, 1989); Handbook of Metal Ligand Heats, 3^{rd} ed. J. J. Christensen & R. M. Izatt (NY: Marcel Dekker, Inc. 1983).

TABLE 1

| Metal Ion | Trientine | Penicillamine |
|---|---|---|
| Copper | 20.05 (Cu²⁺) | 18.18 (Cu¹⁺) |
| Palladium (Pd²⁺) | 39.4 | |
| Mercury (Hg²⁺) | 24.5 | 16.3 |
| Bismuth (Bi³⁺) | 21.9 | |
| Nickel (Ni²⁺) | 13.8 | 10.70 |
| Zinc (Zn²⁺) | 12 | 9.71 |
| Cadmium (Cd²⁺) | 10.6 | 11.55 |
| Cobalt (Co²⁺) | 10.9 | 8.98 |
| Lead (Pb²⁺) | 10.4 | 12.3 |
| Chromium (Cr²⁺) | 7.9 | |
| Iron (Fe²⁺) | 7.76 | |
| Silver (Ag¹⁺) | 7.5 | 12.4 |
| Manganese (Mn²⁺) | 4.90 | |
| Indium (In³⁺) | | 15.33 |
| Thallium (Tl¹⁺) | | 3.58 |

The data in TABLE 1 shows that trientine binds very strongly to palladium, mercury, bismuth, copper, and nickel. The binding of trientine to zinc, cadmium, lead, and cobalt is also fairly strong. TABLE 1 further shows that penicillamine binds copper, mercury and indium very strongly. The binding of penicillamine to cadmium, zinc, lead, nickel, and silver is also fairly strong.

Radionuclides or radioisotopes are atoms with an unstable nucleus. Radionuclides undergo radioactive decay and emit gamma rays and/or subatomic particles, which are harmful to humans and animals. Excessive exposure to radionuclides may cause radiation poisoning, causing damages to organs.

Exemplary radionuclides include americium-241, palladium-103, californium-252, phosphorus-32, cesium-137, plutonium-238, -239, cobalt-60, polonium-210, radium-226, strontium-90 (Sr-90/Y-90), yttrium-90, iridium-192, and uranium-234, -235. Radionuclides are of interest to terrorists as they can be used to build radiological dispersal devices (RDDs) such as dirty bombs to disseminate radioactive materials on a large scale. The invention contemplates the use of trientine and/or penicillamine as a countermeasure to radionuclides, including the above listed radioactive isotopes. A therapeutically effective amount of trientine and/or penicillamine may be administered to affected patients to reverse or mitigate the negative effects of such exposure.

Trientine and/or penicillamine can be used not only to remove radionuclides, but also their breakdown products. For example, yttrium-90 is a radioactive breakdown product of strontium-90, and polonium-210 is a breakdown product of radon-222, which itself comes from uranium-238.

Terrorist attack using radionuclides is a growing concern in the United States. Explosion of RDDs such as dirty bombs produces both radioactive and nonradioactive shrapnel, and radioactive dust, causing radiation contamination, physical injury, burns, as well as panic and fear in densely populated communities. Radiation contamination caused by RDDs may affect regions ranging from a small, localized area such as a street, single building, or city block to a large area up to several square miles. Recognizing the need to quickly respond to a national terrorist attack, the United States has established the Strategic National Stockpile (SNS) Program, to ensure the availability of lifesaving pharmaceuticals, antidotes, and other medical supplies and equipment necessary to counter the negative effects of chemical, radiological and biological pathogens and agents.

The invention contemplates the use of trientine and/or penicillamine in preparing a community or region against radiation contamination on a large scale. The invention includes stockpiling a quantity of dosage forms of trientine sufficient to treat every member of a community within a week or less, preferably within three days or less, more preferably within two days or less, or within one day or less of such exposure to the radiation contamination. The stockpiling of the dosage forms may be sufficient to treat each member of a community ranging from 5,000 to 1 million, or from 10,000 to 1 million, or from 25,000 to 1 million, or from 100,000 to 1 million individuals. In some instances, the stockpiling of the dosage forms may be sufficient to treat each member of a community with 500,000 to 1 million individuals.

People affected with acute exposure to RDDs may be treated on a 28-day course with 4-8 doses per day depending on the degree of the radiation contamination. Each dose may contain, for example, 250 mg trientine hydrochloride in a capsule. Therefore, approximately 112 to 224 doses or capsules are needed for treatment of one individual on a 28-day course. Based on this treatment regimen, approximately 560,000 to 1.1 million doses or capsules of trientine are needed in order to treat each member of a community of about 5,000 individuals. Considering the current trientine production lead time of about 60 days and production capacity in the United States, approximately 560,000 to 1.1 million doses or capsules should be stockpiled to prepare such a community for attack by RDDs such as dirty bombs.

A quantity of dose forms of penicillamine also may be stockpiled and be administered sequentially or concomitantly with trientine. Penicillamine may be stockpiled as combined single dose forms with trientine, or alternatively, as independent dose forms. The stockpiling of penicillamine may be sufficient for treatment of each member of a community ranging from 5,000 to 1 million, or from 10,000 to 1 million, or from 15,000 to 1 million, or from 25,000 to 1 million, or from 100,000 to 1 million individuals. In some instances, the stockpiling of penicillamine dose forms may be sufficient for a community with 500,000 to 1 million individuals.

To treat an affected person with acute exposure, approximately 84-168 doses are needed on a 28-day treatment course with 3-6 doses per day. Based on this treatment regimen, approximately 1,260,000 to 2,520,000 doses of penicillamine are needed for treating each person of an affected community of about 15,000 members. Considering the current penicillamine production lead time of about 73 days and production capacity in the United States, approximately 1,260,000 to 2,520,000 doses should be stockpiled to prepare a community of about 15,000 members for attack by RDDs such as dirty bombs.

The following Table 2 provides the number of dose forms of trientine and penicillamine that are recommended for stockpiling for communities of different size.

TABLE 2

| Community | Trientine | Penicillamine |
|---|---|---|
| (Members) | (Doses, in thousand) | (Doses, in thousand) |
| 5,000 | 560-1,120 | 420-840 |
| 10,000 | 1,120-2,240 | 840-1,680 |
| 15,000 | 1,680-3,360 | 1,260-2,520 |
| 25,000 | 2,800-5,600 | 2,100-4,200 |
| 50,000 | 5,600-11,200 | 4,200-8,400 |
| 100,000 | 11,200-22,400 | 8,400-16,800 |
| 500,000 | 56,000-112,000 | 42,000-84,000 |
| 1,000,000 | 112,000-224,000 | 84,000-168,000 |

In another embodiment, trientine and/or penicillamine is administered in combination with one or more additional decorporation agents used in treating radionucleotide contamination. Such decorporation agents include ammonium chloride, calcium, Ca-DTPA (diethylene triamine pentaacetic acid), calcium gluoconate, dimercaprol, potassium iodide, potassium phosphate, propylthiouracil, Prussian blue, sodium alginate, sodium bicarbonate, sodium phosphate, and Zn-DTPA.

In other embodiments, the additional decorporation agent is a chelating agent that binds to polonium-210. Such chelation agents include, but are not limited to, 2,3-dimercaptopropanol (BAL), 2,3-dimercaptopropane-1-sulfonate (DMPS), 2-(2,3-dimercaptopropoxy)-ethanesulfonate (DMPS, Unithol), 2-(2,3-dimercaptopropoxy)-ethansulphonate (Oxathiol), N-(2,3-dimercaptopropyl)-phthalamidic acid (DMPA), meso-dimercaptosuccinic acid (meso-DMSA), diethyldithiocarbamate (DDTC), meso-2,3-dimercaptosuccinamide (Mi-BDMA), and N,N′-di(2-hydroxyethyl)-ethylenediamine-N,N′-biscarbodithioate (HOEtTTC).

### EXAMPLES

### Example 1

### Decorporation of Cobalt-60

Materials. Chloride stock solution of ⁶⁰Co as ⁶⁰CoCl₂ was diluted with sterile saline solution to adjust to the desired activity and used for the IV dosing. Activity of the dosing solution was determined using automated Wallac 1480 (Perkin Elmer) gamma counter equipped with 3 inch NaI(TI) crystal shielded detector. Penicillamine (CUPRIMINE®) and trientine (SYPRINE®) oral dosing solutions were prepared immediately prior to administration using deionized (DI) water so that a single administered dose would contain the target 15 mg/kg of drug. To prepare dosing solutions, the weighed content of one capsule of penicillamine (0.350 g) or trientine (0.270 g; stored in refrigerator at 4-6° C. prior to use) was dissolved in 20 mL of DI water and filtered. Consequently, 2.5 and 3.5 mL aliquots of penicillamine and trientine solutions, respectively, were diluted to 6 mL with DI water. The 0.5 mL of the resulting solution contained 5.0 or 5.2 mg/mL of penicillamine or trientine, respectively, were administered via oral gavage to the Male Wistar-Han rats.

Animals. Male Wistar-Han rats were obtained from Charles River Breeding Laboratory (Raleigh, N.C.) with indwelling jugular vein cannula. Animals were provided food and water ad libitum during acclimatization. The light cycle was 12 hour light/12 hour dark and relative humidity and temperature maintained at 50±15% and 22±2° C. All animal use protocols were approved by the Institutional Animal Care and Use Committee at Battelle, Pacific Northwest Division and studies were performed according to the “Guide for the Care and Use of Laboratory Animals” (National Research Council, Washington D.C., 1996).

Dosing regimen. Animals were restricted from food overnight prior to exposure. At the time of exposure, groups of animals (N=6) received a single intravenous (IV) injection (0.2 mL) of ⁶⁰Co solution at 14.0±0.6 KBq dose in sterile saline via an indwelling jugular vein cannula. Immediately following IV injection, two groups of animals received an oral gavage dose of 0.5 mL penicillamine or trientine aqueous solution at a single target daily dose of 15 mg/kg. The actual doses delivered were calculated based on the individual animal weights (Table 3). One group of animals was dosed with the radionuclide without subsequent administration of the chelation material to serve as control group. Following dosing, animals were placed in Nalgene metabolism cages for separate collection of urine and feces. Animals were restricted from food for 1 hr after dosing with the penicillamine or trientine. Animals were sacrificed 48 hr post radionuclide administration. At sacrifice, blood and tissues (liver, kidney, spleen, gastrointestinal tract, muscle, bone, bone marrow, and lung) were collected, weighed and counted for radioactivity using a Wallac 1480 gamma counter. Gamma count data were normalized to percent administered dose after adjusting the grams of tissue collected for total organ mass.

TABLE 3

| | | | | | | | | | | Time (hrs) |
|---|---|---|---|---|---|---|---|---|---|---|
| | Animal | | Co-60 Exposure | | | Drug Administration | | | | from Co-60 |
| | | Weigh | | Dose | No. of | | | | No. of | Exposure |
| Group | ID | (grams) | Route | KBq | doses | Drug | Route | Dose mg/kg | doses | To Sacrifice |
| Control | 1 | 177.0 | IV | 14 | 1 | None | | n/a | | 48 |
| | 2 | 175.1 | | | | | | | | |
| | 3 | 177.2 | | | | | | | | |
| | 4 | 167.5 | | | | | | | | |
| | 5 | 185.4 | | | | | | | | |
| | 6 | 178.1 | | | | | | | | |
| penicillam | 7 | 173.4 | IV | 14 | 1 | penicillamine | Oral | 14.4 | 1 | 48 |
| | 8 | 170.3 | | | | | | 14.7 | | |
| | 9 | 173.7 | | | | | | 14.4 | | |
| | 10 | 177.7 | | | | | | 14.1 | | |
| | 11 | 163.9 | | | | | | 15.3 | | |
| | 12 | 187.2 | | | | | | 13.4 | | |
| trientine | 13 | 176.9 | IV | 14 | 1 | trientine | Oral | 14.7 | 1 | 48 |
| | 14 | 173.7 | | | | | | 15.0 | | |
| | 15 | 163.9 | | | | | | 15.9 | | |
| | 16 | 173.6 | | | | | | 15.0 | | |
| | 17 | 182.1 | | | | | | 14.3 | | |
| | 18 | 159.0 | | | | | | 16.4 | | |

Data statistical evaluation. Each data group was subjected to Dixon's Q-test to evaluate for potential outliers. In this test Q parameter of 0.625 (N=6, 95% confidence level, a=0.05) was used (1). For each tissue, a preliminary F-test (95% confidence level, a=0.05) for the equality of variances of the control and each treatment group was performed using Excel software. If the calculated probability p value was less than 0.05, the variances were assumed to be not equal. Based on this information, “T-test: Two-Sample Assuming Unequal Variances” or “T-test: Two-Sample Assuming Equal Variances” (95% confidence level, a=0.05) was performed using Excel software. The calculated p-value less than 0.05 provides evidence to reject the null hypothesis of equal means. The obtained statistical parameters listed in Table 4 served as guidelines for data evaluation (Table 5; calculation assumes that total skeleton, blood, or muscle is approximately 7.3, 6, or 40% of the body weight of the animal, respectively; Co-60 in skeleton is calculated based on the femur data—R. P. Brown, M. D. Delp, S. L. Undstedt, L. R. Rhomberg, and R. P. Beliles, Physiological Parameter Values for Physiologically Based Pharmacokinetic Models. Toxicology and Industrial Health, 1997, 13(4): 407-484).

TABLE 4
Summary of the statistical data evaluation.

| | | | Q-test | | | | | T-test | | | |
|---|---|---|---|---|---|---|---|---|---|---|---|
| | Animal | | # of | | F-test | | | | | | Equal |
| Tissue | group | N | outliers | Final N | F | p | Variance | DF | t | p | means |
| Total | Control | 6 | 0 | 6 | | | | | | | |
| skeleton | penicillamine | 6 | 0 | 6 | 1.77 | 0.27 | Equal | 10 | 2.65 | 0.024 | No |
| | trientine | 6 | 0 | 6 | 1.06 | 0.47 | Equal | 10 | 1.68 | 0.12 | Yes |
| Kidney | Control | 6 | 0 | 6 | | | | | | | |
| | penicillamine | 6 | 0 | 6 | 9.40 | 0.014 | Unequal | 6 | 4.37 | 0.002 | No |
| | trientine | 6 | 0 | 6 | 22.6 | 0.0019 | Unequal | 5 | 1.05 | 0.34 | Yes |
| Liver | Control | 6 | 1 | 5 | | | | | | | |
| | penicillamine | 6 | 0 | 6 | 1.13 | 0.44 | Equal | 9 | 6.58 | 0.0001 | No |
| | trientine | 6 | 0 | 6 | 0.32 | 0.15 | Equal | 9 | 0.11 | 0.91 | Yes |
| Blood | Control | 6 | 0 | 6 | | | | | | | |
| | penicillamine | 6 | 1 | 5 | 17.7 | 0.0008 | Unequal | 6 | 0.071 | 0.95 | Yes |
| | trientine | 6 | 0 | 6 | 0.47 | 0.21 | Equal | 10 | −2.21 | 0.049 | No |
| Muscle | Control | 6 | 0 | 6 | | | | | | | |
| | penicillamine | 6 | 0 | 6 | 3.80 | 0.084 | Equal | 10 | 2.51 | 0.031 | No |
| | trientine | 6 | 0 | 6 | 2.18 | 0.21 | Equal | 10 | −0.55 | 0.59 | Yes |
| Lung | Control | 6 | 0 | 6 | | | | | | | |
| | penicillamine | 6 | 0 | 6 | 1.43 | 0.35 | Equal | 10 | −0.76 | 0.46 | Yes |
| | trientine | 6 | 0 | 6 | 3.29 | 0.11 | Equal | 10 | 0.035 | 0.97 | Yes |
| Spleen | Control | 6 | 0 | 6 | | | | | | | |
| | penicillamine | 6 | 0 | 6 | 2.69 | 0.15 | Equal | 10 | −1.43 | 0.18 | Yes |
| | trientine | 6 | 0 | 6 | 0.74 | 0.38 | Equal | 10 | −1.32 | 0.21 | Yes |
| Stoma | Control | 6 | 1 | 5 | | | | | | | |
| | penicillamine | 6 | 0 | 6 | 1.35 | 0.37 | Equal | 9 | 3.77 | 0.004 | No |
| | trientine | 6 | 0 | 6 | 1.86 | 0.25 | Equal | 9 | 0.32 | 0.76 | Yes |
| Intesti | Control | 6 | 0 | 6 | | | | | | | |
| | penicillamine | 6 | 0 | 6 | 9.88 | 0.012 | Unequal | 6 | 2.42 | 0.050 | No |
| | trientine | 6 | 0 | 6 | 11.4 | 0.0092 | Unequal | 6 | 2.31 | 0.060 | Yes |

TABLE 5
Tissue distribution of IV administered Co-60 in Wistar-Han rats:
effect of oral treatment with penicillamine and trientine.

| | Co-60 Dose % for animal group | | | | |
|---|---|---|---|---|---|
| | | Co-60 + | Co-60 + | % Reduction for | |
| Tissue | Co-60 Control | penicillamine | trientine | penicillamine | trientine |
| Total Skeleton | 0.63 ± 0.23 | 0.32 ± 0.17 | 0.41 ± 0.22 | 49 | 35 |
| Bone Marrow | Below detection | Below detection | Below detection | | |
| Kidney | 0.71 ± 0.21 | 0.31 ± 0.07 | 0.62 ± 0.04 | 56 | 13 |
| Liver | 2.02 ± 0.27 | 0.99 ± 0.25 | 2.00 ± 0.47 | 51 | 0 |
| Blood | 0.091 ± 0.058 | 0.089 ± 0.014 | 0.18 ± 0.08 | 0 | −98 |
| Muscle | 1.03 ± 0.35 | 0.63 ± 0.18 | 1.13 ± 0.24 | 39 | 0 |
| Lung | 0.13 ± 0.08 | 0.16 ± 0.07 | 0.13 ± 0.05 | 0 | 0 |
| Spleen | 0.049 ± 0.011 | 0.056 ± 0.007 | 0.058 ± 0.013 | 0 | 0 |
| Stomach | 0.075 ± 0.016 | 0.041 ± 0.014 | 0.072 ± 0.012 | 45 | 0 |
| Intestine | 1.76 ± 0.79 | 0.94 ± 0.25 | 0.98 ± 0.23 | 47 | 44 |

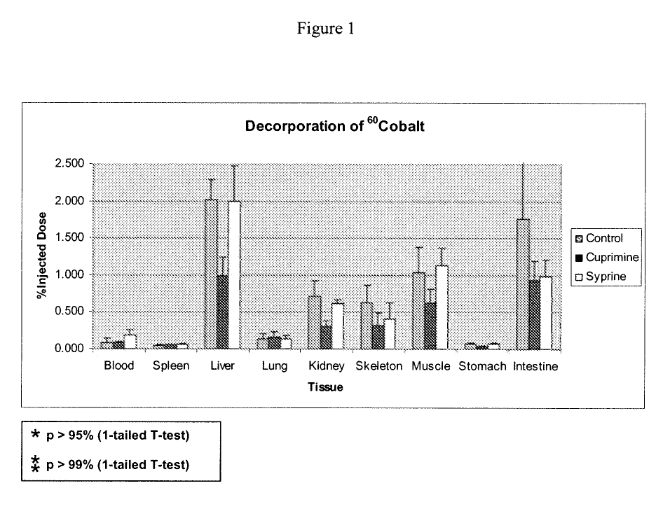

Co-60 elimination was monitored for 2 days following a single IV injection. The results are presented in FIG. 1 and Tables 4-5. The combined urine and fecal excretion expressed as Co-60 radioactivity (disintegrations per minute—DPM) per gram of total excreta was the highest at day 1 post exposure and decreased 3.6 times by day 2. The predominant route of excretion was via the urine, with about 47 and 9% of the administered radioactivity excreted in the urine within the first and second day post exposure, respectively. In comparison, fecal elimination accounted for approximately 9 and 4.4% of the administered radioactivity at the same time intervals. Administration of penicillamine or trientine appeared to accelerate total Co-60 excretion, however this result was not statistically significant.

All tissues (except bone marrow) collected post-mortem at day 2 post Co-60 exposure were found to contain measurable amounts of radioactivity (Table 5), with the highest levels in the liver followed by muscle, intestine, kidney, and skeleton. The percent of administered radioactivity for the whole skeleton was calculated based on the femur data under the assumption that femur is representative of the bone as a whole. Lung, spleen, and stomach tissues were found to contain smaller amounts of radioactivity. Blood had residual radioactivity at day 2 following IV injection (Table 5).

Penicillamine or trientine were administered orally immediately after IV dosing with Co-60. The actual doses (mg of drug per kg body weight) are listed in Table 3. The mean dose was 14.6±0.6 and 15.2±0.8 for penicillamine and trientine, respectively. Penicillamine did not change the urinary elimination of Co-60 although administration did increase fecal elimination of Co-60 by about 3% at day 1 post exposure, so that the total excretion was slightly increased. Trientine noticeably enhanced urinary excretion at day 1. Fecal elimination, however, decreased, and the overall effect on excretion was similar to that of penicillamine.

For animals treated with oral penicillamine, significant reductions in the distribution of radioactivity to tissues were observed. For example, administration of penicillamine resulted in 51-56% reduction in the fraction of administered dose in the skeleton (based on femur data), liver and kidney (Table 5) upon administration of only a single dose of penicillamine. Similarly, lower levels of the percent administered dose were observed in stomach and muscle with the corresponding reductions of 45 and 39%, respectively. Penicillamine slightly decreased blood levels of Co-60.

Although administration of trientine resulted in elevated Co-60 in blood (Table 5), trientine decreased levels of Co-60 in the skeleton tissue by 35% and intestine by 44%, and appeared to maintain Co-60 in circulation, which may be of value to protect organs into which it would otherwise deposit. Use of trientine may thus be advantageous to patients who are intolerant to D-penicillamine. Additionally, concomitant or sequential use of penicillamine and trientine may remove more Co-60 than use of the single agents. Trientine may help maintain Co-60 in the circulation, from which it is more easily removed by penicillamine than when it is deposited in the organs.

### Example 2

### Decorporation of Polonium-210

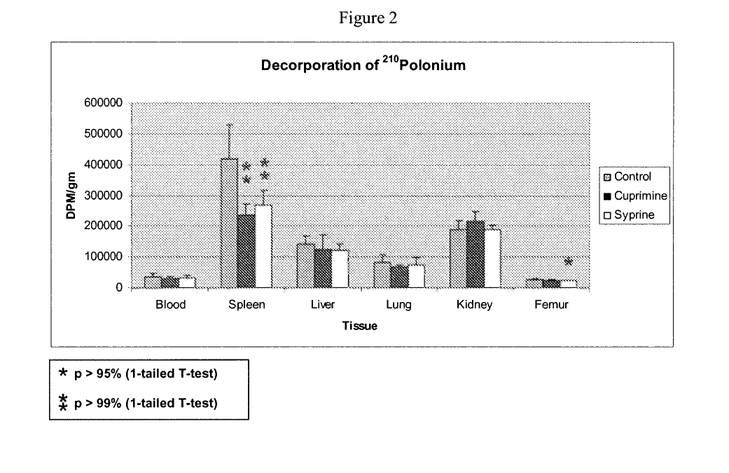

Animals received a single IV dose of Po-210 (approximately 90 kBq/animal, followed by oral administration of penicillamine or trientine (15 mg/kg). The drug dosing was repeated in 24 h intervals, total number of doses was 5. Animals were fasted 1 h prior and 1 h post drug administration. On day 6 post radiation exposure animals were sacrificed, and tissues collected, weighed, and processed for analysis. The relative alpha activity of the samples was determined by liquid scintillation counting (LSC) using a Packard Tri-Carb 2260XL instrument. Samples of blood, feces, and liver collected from animals exposed to Po-210 were digested using hydrogen peroxide and concentrated nitric acid solutions at room temperature. The digested sample was diluted with distilled water to reduce the nitric acid to approximately 1 M. An aliquot of the resulting solution was added to Ultima Gold XR liquid scintillation cocktail (Packard BioScience, Meriden, Conn.), counted and corrected for possible quenching. As shown in Table 6 and FIG. 2, penicillamine reduced polonium-210 in the spleen femur and lung, while trientine reduced levels in the spleen, femur and liver.

TABLE 6
Tissue distribution of IV administered Po-210 in Wistar-Han rats:
effect of oral treatment with penicillamine and trientine.

| | Po-210 dpm/gm for animal group | | | | |
|---|---|---|---|---|---|
| | | Po-210 + | Po-210 + | % Reduction for | |
| Tissue | Po-210 Control | penicillamine | trientine | penicillamine | trientine |
| Femur | 26479 ± 3212 | 22969 ± 4055 | 22415 ± 2148 | 13 | 15 |
| Kidney | 188050 ± 29333 | 216172 ± 31993 | 187883 ± 15839 | −15 | 0 |
| Liver | 141127 ± 26065 | 124648 ± 47423 | 120883 ± 21221 | 12 | 14 |
| Blood | 34928 ± 11610 | 28482 ± 6375 | 33273 ± 7289 | 18 | 0 |
| Lung | 83673 ± 21793 | 67342 ± 7895 | 74512 ± 22281 | 20 | 11 |
| Spleen | 419754 ± 110289 | 236736 ± 35488 | 269303 ± 47082 | 44 | 36 |

### Example 3

### General Method for Assessing Decorporation of Other Metals

Penicillamine (CUPRIMINE®) and trientine (SYPRINE®) oral dosing solutions are prepared according to Example 1. Male Wistar-Han rats are restricted from food overnight prior to exposure and given a single intravenous (IV) injection of a metal contaminant solution containing, for example, strontium, cesium, radium, palladium, iridium, uranium, plutonium, americium, curium, californium, and/or combinations thereof or isotopes thereof. Immediately following IV injection, two groups of animals are given an oral gavage dose of 0.5 mL penicillamine or trientine aqueous solution at a single target daily dose of 15 mg/kg. Animals are sacrificed 48 hr post radionuclide administration. At sacrifice, blood and tissues (liver, kidney, spleen, gastrointestinal tract, muscle, bone, bone marrow, and lung) are collected, weighed and analyzed for presence of the metal contaminant.

From the foregoing it will be appreciated that, although specific embodiments of the invention have been described herein for purposes of illustration, various modifications may be made without deviating from the spirit and scope of the invention claimed in the claims.

We claim:

1. A method of treating a mammal suffering from one or more negative effects of overexposure to a metal contaminant comprising administering to the affected mammal a therapeutically effective amount of trientine or a pharmaceutically acceptable salt or solvate thereof, which metal contaminant includes at least one metal selected from the group consisting of a metal from Group IA, IIA, VIIB, VIIB, VIII, IB, IIB, IIIA, IVA, VA, VIA, Lanthanide Series, and Actinide Series of the periodic table, wherein said at least one metal is other than copper.

2. The method of claim 1 wherein said at least one metal is selected from the group consisting of mercury, nickel, bismuth, palladium, zinc, cadmium, lead, cobalt, chromium, iron, silver, and cesium.

3. The method of claim 1 wherein said at least one metal is palladium.

4. The method of claim 1 wherein said at least one metal is cobalt.

5. The method of claim 1 wherein said at least one metal is a radioactive isotope.

6. The method of claim 2 wherein said at least one metal is a radioactive isotope.

7. The method of claim 1 wherein said at least one metal is a radioactive isotope of a metal selected from the group consisting of americium, californium, cobalt, iridium, palladium, plutonium, polonium, and uranium.

8. The method of claim 7 wherein said at least one metal is polonium.

9. The method of claim 1 wherein said administration is carried out enterally.

10. The method of claim 1 wherein said administration is carried out parenterally.

11. The method of claim 1 wherein said trientine or a pharmaceutically acceptable salt or solvate thereof is administered in a dose ranging from about 4 mg of free base per kg of mammal per day to about 25 mg of free base per kg of mammal per day.

12. The method of claim 1 wherein said administration is carried out sequentially or concomitantly with the administration of an effective amount of penicillamine or a pharmaceutically acceptable salt, ester, or solvate thereof.

13. The method of claim 12 wherein said penicillamine or a pharmaceutically acceptable salt, ester, or solvate thereof is administered in a dose ranging from about 2 mg of penicillamine per kg of mammal per day to about 30 mg of penicillamine per kg of mammal per day.

14. The method of claim 12 wherein the affected mammal is overexposed to at least two metals.

15. The method of claim 14 wherein said at least two metals includes indium.

16. The method of claim 12 wherein effective amounts of trientine hydrochloride and d-penicillamine are co-administered.

17. A method of treating a community of individuals against the negative effects of overexposure to metal contamination on a large scale comprising providing a quantity of dosage forms of trientine or a pharmaceutically acceptable salt or solvate thereof sufficient to treat within a week or less of said overexposure every member of a community numbering between 5,000 and 1,000,000 individuals.

18. The method of claim 17 wherein a quantity of dosage forms of penicillamine or a pharmaceutically acceptable salt, ester, or solvate thereof sufficient to treat within a week or less of said overexposure every member of a community numbering between 15,000 and 1,000,000 individuals is also provided.

19. The method of claim 18 wherein said dosage forms of trientine or a pharmaceutically acceptable salt or solvate thereof and said dosage forms of penicillamine or a pharmaceutically acceptable salt, ester, or solvate thereof are provided in combined single dosage forms.

20. The method of claim 17 wherein said quantity of dosage forms of trientine or a pharmaceutically acceptable salt or solvate thereof is provided sufficient to treat within three days or less of said overexposure every member of the community.

21. The method of claim 17 wherein said quantity of dosage forms of trientine or a pharmaceutically acceptable salt or solvate thereof is provided sufficient to treat within two days or less of said overexposure every member of the community.

22. The method of claim 17 wherein said quantity of dosage forms of trientine or a pharmaceutically acceptable salt or solvate thereof is provided sufficient to treat within one day or less of said overexposure every member of the community.

23. The method of claim 17 wherein said quantity of dosage forms of trientine or a pharmaceutically acceptable salt or solvate thereof is provided sufficient to treat within a week or less of said overexposure every member of a community numbering between 15,000 and 1,000,000 individuals.

24. The method of claim 17 wherein said quantity of dosage forms of trientine or a pharmaceutically acceptable salt or solvate thereof is provided sufficient to treat within a week or less of said overexposure every member of a community numbering between 100,000 and 1,000,000 individuals.

25. The method of claim 17 wherein said quantity of dosage forms of trientine or a pharmaceutically acceptable salt or solvate thereof is provided sufficient to treat within a week or less of said overexposure every member of a community numbering between 500,000 and 1,000,000 individuals.

26. A method of preparing a community against the negative effects of overexposure to metal contamination on a large scale comprising stockpiling a quantity of dosage forms of trientine or a pharmaceutically acceptable salt or solvate thereof sufficient to treat within a week or less of said overexposure every member of a community numbering between 5,000 and 1,000,000 individuals.

27. The method of claim 26 wherein a quantity of dosage forms of penicillamine or a pharmaceutically acceptable salt, ester, or solvate thereof sufficient to treat within a week or less of said overexposure every member of a community numbering between 15,000 and 1,000,000 individuals is also stockpiled.

28. The method of claim 27 wherein said dosage forms of trientine or a pharmaceutically acceptable salt or solvate thereof and said dosage forms of penicillamine or a pharmaceutically acceptable salt, ester, or solvate thereof are stockpiled in combined single dosage forms.

29. The method of claim 26 wherein said quantity of dosage forms of trientine or a pharmaceutically acceptable salt or solvate thereof is stockpiled sufficient to treat within three days or less of said overexposure every member of the community.

30. The method of claim 26 wherein said quantity of dosage forms of trientine or a pharmaceutically acceptable salt or solvate thereof is stockpiled sufficient to treat within two days or less of said overexposure every member of the community.

31. The method of claim 26 wherein said quantity of dosage forms of trientine or a pharmaceutically acceptable salt or solvate thereof is stockpiled sufficient to treat within one day or less of said overexposure every member of the community.

32. The method of claim 26 wherein said quantity of dosage forms of trientine or a pharmaceutically acceptable salt or solvate thereof is stockpiled sufficient to treat within a week or less of said overexposure every member of a community numbering between 15,000 and 1,000,000 individuals.

33. The method of claim 26 wherein said quantity of dosage forms of trientine or a pharmaceutically acceptable salt or solvate thereof is stockpiled sufficient to treat within a week or less of said overexposure every member of a community numbering between 100,000 and 1,000,000 individuals.

34. The method of claim 26 wherein said quantity of dosage forms of trientine or a pharmaceutically acceptable salt or solvate thereof is stockpiled sufficient to treat within a week or less of said overexposure every member of a community numbering between 500,000 and 1,000,000 individuals.

35. The method of claim 12 wherein said trientine or a pharmaceutically acceptable salt or solvate thereof and said penicillamine or a pharmaceutically acceptable salt, ester, or solvate thereof are concomitantly administered in a single combination dosage form.

36. A method of treating a mammal suffering from one or more negative effects of overexposure to a metal contaminant comprising sequentially or concomitantly administering to the affected mammal a therapeutically effective amount of trientine or a pharmaceutically acceptable salt or solvate thereof and penicillamine or a pharmaceutically acceptable salt, ester, or solvate thereof, which metal contaminant includes at least one radioactive isotope of a metal selected from the group consisting of americium, californium, cobalt, curium, iridium, palladium, plutonium, polonium, radium, strontium, and uranium.

37. The method of claim 36 wherein the radioactive isotope is cobalt-60 or polonium-210 or a combination thereof.

38. A pharmaceutical dosage form comprising an effective amount of trientine or its pharmaceutically acceptable salt or solvate and an effective amount of penicillamine or its pharmaceutically acceptable salt, ester or solvate and optionally one or more pharmaceutically acceptable carriers.

39. A method of treating a mammal suffering from one or more negative effects of overexposure to polonium, comprising administering to the affected mammal a therapeutically effective amount of penicillamine or a pharmaceutically acceptable salt, ester, or solvate thereof.

40. The method of claim 39 wherein said administration is carried out enterally.

41. The method of claim 39 wherein said administration is carried out parenterally.

42. The method of claim 39 wherein said penicillamine or a pharmaceutically acceptable salt or solvate thereof is administered in a dose ranging from about 2 mg of free base per kg of mammal per day to about 30 mg of free base per kg of mammal per day.

43. The method of claim 14 wherein said at least two metals includes polonium.
